(19) United States
(12) Patent Application Publication
(10) Pub. No.: US 2026/0283207 A1
(43) Pub. Date: Sep. 24, 2026

(54) Mycelium Filaments

(71) Applicant: Coöperatie Koninklijke Cosun U.A., Breda (NL)

(72) Inventors: Hans de Bruijn, Dinteloord (NL); Michaël Johannes Jacobus Litjens, Dinteloord (NL)

(73) Assignee: Coöperatie Koninklijke Cosun U.A., Breda (NL)

(21) Appl. No.: 19/490,126

(22) PCT Filed: Jun. 10, 2024
(86) PCT No.: PCT/EP2024/065937

§ 371 Date: Dec. 4, 2025

(30) Foreign Application Priority Data
Jun. 9, 2023 (EP) 23178463.8

(51) Int. Cl. A23L 31/00; A23L 33/22; C12N 1/145; C12R 1/69; C12R 1/845

(52) U.S. Cl. CPC A23L 31/00; A23L 33/22; C12N 1/145; C12R 2001/69; C12R 2001/845

(57) ABSTRACT

The invention pertains to a composition comprising fiber-reinforced mycelium filaments comprising fungal mycelium and a plant-based fiber, wherein the plant-based fiber comprises sugar beet pulp.

The present invention relates to mycelium filaments.

Mycelium filaments, and in particular edible mycelium filaments, are well known in the art. Mainly such filaments are used as meat or fish replacements. Many authors describe the solid state fermentation of seeds, spent grain or stale bread with edible filamentous fungi such as Gmoser et al (2020; DOI: 10.1080/21655979.2020.1768694). Further solid state fermentation processes to produce food products are described in CA 3,130,259, WO 2022/229072, WO 2020/232347, WO 2022/079452 and WO 2022/034092.

Alternatively, submerged or liquid fermentation processes are used to produce a pure mycelium from sugar or starch substrates and/or to improve the protein content of substrates such as spent grain as is disclosed in Parchami et al (2021, DOI: 10.15376/biores.16.1.1686-1701). Also, in WO 2022/157326 and U.S. Pat. No. 8,343,741 food products from edible fungal filaments are produced using submerged fermentation. The main focus of these disclosures is the growth of the edible fungal filaments and the resulting fungal product is reported to have a structure close to meat. There is still a need to alter the properties of these fungal filaments and improve the sensorial and textural properties.

The objective of the present invention is to provide novel mycelium filament-containing compositions.

The invention pertains to a composition comprising fiber-reinforced mycelium filaments comprising fungal mycelium and a plant-based fiber, wherein the plant-based fiber comprises sugar beet pulp. The mycelium filaments comprise plant-based fibers, in particular the sugar beet pulp, which form part of the filaments and/or are embedded into the filament structure. The presence of the plant-based fiber generally leads to improved physical and/or chemical properties of the mycelium filaments, such as improved elasticity and strength of the inventive filaments. The texture of the inventive filaments is different from conventional filaments without plant-based fiber, in particular toughness and/or firmness is improved. Moreover, juiciness, consistency and/or sensory properties of the food products in which the inventive filaments are contained are improved. In addition, the inventive filaments bring about a meat-like or fish-like consistency and are suitable as meat or fish replacement. The inventors have found that the invention can be applied to a wide variety of fungal mycelium, in particular edible fungal filaments. Moreover, a wide variety of plant-based fibers can be suitably used in the filaments of the invention. A further advantage is that the inventive filaments show better sedimentation compared to conventional filaments without the plant-based fiber, rendering their production and further processing easier. The compositions of the invention can also be produced using plant-based fiber which contain nutrients for the growth of the fungal mycelium. Examples of such plant-based fibers include sugar beet pulp with 2 to 20 wt % of sucrose, brewer's spent grain fibers comprising gluten and/or other proteins, and pea-derived fiber with starch. The nutrient-containing plant-based fiber may lead to nutrient provision where nutrients added to the medium may experience mass transfer limitations while reaching the growth site. This in turn may increase hyphal penetration of the plant fiber, increase growth rate, reduce the formation of chlamydospores and additionally may reduce operation costs.

In one embodiment of the invention, the fungal mycelium are mycelium filaments and the plant-based fiber from part of the mycelium filaments and/or are embedded into the filament structure. With the wording “part of the mycelium filaments” is meant that the fungal filaments have grown through the plant-based fibers and/or are chemically connected to the fibers. With the wording “embedded into the filament structure” is meant that the fibers are surrounded by the fungal filaments in such a way that the fibers cannot move separately from the fungal filaments; there is no chemical connection between the filaments and the fibers. This can be determined using scanning electron microscopy (SEM) or light microscopy.

In one embodiment of the invention, the inventive composition comprises at least 1 wt % protein. Preferably, the inventive composition comprises at least 2 wt % protein, more preferably at least 5 wt % protein, even more preferably at least 10 wt % protein and most preferably at least 15 wt % protein, and preferably at most 50 wt % protein, more preferably at most 40 wt % protein and most preferably at most 30 wt % protein, based on the total weight of the composition. Various methods have been described in literature to determine the protein content. For the purposes of this application, the Kjeldahl method is used to determine the nitrogen content, which is then converted to protein content. The Kjeldahl is well established and well known to the person skilled in the art. In this application the Kjeldahl method is performed by hydrolyzing a sample using H₂SO₄ at 420° C. for 2 hours, during which the proteins will be converted to ammonia. The generated ammonia is distilled off and the amount of nitrogen is measured by titration. The amount of protein is calculated by multiplying the nitrogen content by the conversion factor of 6.25 (nitrogen to protein factor).

In one embodiment of the invention, the inventive composition comprises at least 10 wt % crude fiber. Preferably, the inventive composition comprises at least 15 wt % crude fiber, more preferably at least 20 wt % crude fiber, even more preferably at least 25 wt % crude fiber and most preferably at least 30 wt % crude fiber, and preferably at most 50 wt % crude fiber, more preferably at most 40 wt % crude fiber and most preferably at most 30 wt % crude fiber, based on the total weight of the composition. The crude fiber is determined using the ISO 68651:2000 method.

In one embodiment of the invention, the inventive composition comprises at least 10 wt % insoluble HMWDF. Preferably, the inventive composition comprises at least 15 wt % insoluble HMWDF, more preferably at least 20 wt % insoluble HMWDF, even more preferably at least 25 wt % insoluble HMWDF and most preferably at least 30 wt % insoluble HMWDF, and preferably at most 50 wt % insoluble HMWDF, more preferably at most 40 wt % insoluble HMWDF and most preferably at most 30 wt % insoluble HMWDF, based on the total weight of the composition. The insoluble high molecular weight dietary fibers (insoluble HMWDF) can be determined using the standard method AOAC 2011.25 dietary fiber analysis, with which the insoluble HMWDF, the soluble HMWDF and the low molecular weight dietary fibers (LMWDF) can be determined.

In one embodiment of the invention, the inventive composition comprises at least 10 wt % water. Preferably, the inventive composition comprises at least 15 wt % water, more preferably at least 20 wt % water, even more preferably at least 25 wt % water and most preferably at least 30 wt % water, and preferably at most 80 wt % water, more preferably at most 60 wt % water and most preferably at most 50 wt % water, based on the total weight of the composition.

In a further embodiment of the invention, the composition of the invention comprises at most 100 ppm gluten. Preferably, the inventive composition comprises at most 50 ppm gluten, more preferably at most 40 ppm gluten, even more preferably at most 30 ppm, and most preferably at most 20 ppm, and preferably at least 10 ppb gluten, more preferably at least 500 ppb, even more preferably at least 1 ppm and most preferably at least 2 ppm. In one embodiment, the composition is gluten-free, i.e. the composition comprises less than 20 ppm. Gluten content was determined using AOAC 2012.01.

In one embodiment of the invention, the composition of the invention comprises at most 5 wt % ethanol. Preferably, the inventive composition comprises at most 2 wt % ethanol, more preferably at most 1 wt % ethanol, even more preferably at most 0.5 wt % ethanol and most preferably at most 0.1 wt % ethanol, and preferably at most 0.05 wt % ethanol, more preferably at least 0.0001 wt % ethanol and most preferably at least 0.001 wt % ethanol, based on the total weight of the composition. The fermentation conditions are generally chosen such that no or hardly any ethanol is formed, thereby rendering mycelium filaments of the invention with (very) low amounts of ethanol. In one embodiment, ethanol is not present in the mycelium filaments of the invention. Alternatively or additionally, ethanol can be removed through evaporation and/or dissolution in order to reach the desired low ethanol levels in the inventive filaments.

The fungal mycelium can be any fungal mycelium known in the art. In one embodiment, the fungal mycelium is an edible fungal mycelium. In another embodiment, the fungal mycelium does not form fruiting bodies. Preferably, the fungal mycelium is derived from species within Ascomycota and Zygomycota phyla. More preferably, the fungal mycelium is selected from Rhizopus, Neurospora, Aspergillus, Trichoderma, Pleurotus, Ganoderma, Inontus, Cordyceps, Tuber, Fusarium, Pennicillium, Xylana, Monascus, Rhizomucor, Trametes, and combinations thereof. Even more preferably, the fungal mycelium is selected from Rhizopus oryzae, Rhizopus microsporus, Rhizopus oligosporus, Neurospora crassa, Neurospora intermedia, Neurospora sitophila, Aspergillus oryzae, Aspergillus awamori, Cordyceps militaris, Codryceps sinensis, Tuber melanosporum, Tuber magnatum, Fusarium graminareum, Fusarium venenatum, Rhizomucor pusillus, Rhizomucor miehei, Pennicillium camemberti and Xylaria hypoxion. Even more preferably, the the fungal mycelium is fungal mycelium selected from the group consisting of Rhizopus oryzae, Aspergillius oryzae and pleurotus pulmonarius. Even more preferably, the fungal mycelium is selected from Rhizopuzs oryzae, Fusarium venenatum and Apergillus oryzae. Most preferably, the fungal mycelium is fungal mycelium of Rhizopus oryzae. It is also contemplated to use two or more fungal mycelia.

In one embodiment of the invention, the composition of the invention comprises at least 20 wt % of the fungal mycelium. Preferably, the inventive composition comprises at least 30 wt % fungal mycelium, more preferably at least 40 wt % fungal mycelium, even more preferably at least 50 wt % fungal mycelium and most preferably at least 60 wt % fungal mycelium, and preferably at most 99 wt % fungal mycelium, more preferably at most 98 wt % fungal mycelium and most preferably at most 95 wt % fungal mycelium, based on the total weight of the fiber-reinforced mycelium filaments.

In yet another embodiment, the fungal mycelium comprises at most 1 chlamydospore per mycelium filament. Preferably, at most 0.5 chlamydospores per mycelium filament, more preferably at most 0.2 chlamydospores per mycelium filament and most preferably at most 0.1 chlamydospores per mycelium filament. The wording “at most 0.5 chlamydospores per mycelium filament” means that on average less than 1 chlamydospore per 2 mycelium filaments is observed using light microscopy or SEM.

The plant-based fiber can be any plant-based fiber known in the art. Preferably, the plant-based fiber is edible. In another embodiment, the plant-based fiber is a dietary fiber. Generally, plant-based fiber comprises cellulose, hemicellulose, pectin and/or lignin, which can be determined using conventional analytical techniques. The term “NDF” is known as “Neutral Detergent Fiber”, which includes cellulose, hemicellulose and lignin. The term “ADF” refers to “Acid Detergent Fiber” and includes cellulose and lignin. For the determination of the amount of lignin the term “ADL” is used which refers to “Acid Detergent Lignin”. The ADF and ADL values were determined according to the standard method NEN-ISO 13906:2008, and the NDF value was determined according to the standard method NEN-ISO 16472:2006. Alternatively or additionally, the high and low molecular weight dietary fibers can be determined using conventional techniques. Examples of suitable plant-based dietary fibers include brewer spent grain; cereal spent grain; cereal fibers; sugar beet pulp; fruit fiber such as fibers derived from apples and pears; vegetable fibers such as fibers derived from lettuce and legumes such as peas and soybeans; and nuts such as almond.

In one embodiment of the invention, the plant-based fiber comprises nutrients, preferably nutrients suitable for growing the fungal filaments. Examples of such nutrients include sugar including glucose and sucrose, starch, proteins and combinations of one or more nutrients. In one embodiment of the invention, the plant-based fiber comprises at least 1 wt % of the nutrient. Preferably, the plant-based fiber comprises at least 2 wt % nutrient, more preferably at least 5 wt % nutrient, even more preferably at least 10 wt % nutrient and most preferably at least 15 wt % nutrient, and preferably at most 60 wt % nutrient, more preferably at most 50 wt % nutrient and most preferably at most 40 wt % nutrient, based on the total weight of the plant-based fiber.

The inventive composition comprises plant-based fiber comprising sugar beet pulp. It was found that the sugar beet pulp can be partly consumed during fermentation, and consequently serves as a nutrient. In a preferred embodiment, the sugar beet pulp has a water holding capacity of at least 14 times the dry weight of the sugar beet pulp. More details of the properties and further embodiments of the sugar beet pulp as well as the process to obtain the sugar beet pulp can be found in PCT/EP2022/083633 and these details and embodiments are included in this specification. In one embodiment, the sugar beet pulp comprises intact cells. Such intact cells may provide a better water holding capacity of the resulting fiber-reinforced mycelium composition, and consequently an improved juiciness.

Advantageously, sucrose remaining in the pulp structure after sucrose extraction and pulp sterilization will be released gradually during the mycelium production fermentation, either through diffusion into the broth or hydrolysis by the fungal hypae upon penetration of the beet fiber. Such effect reduces the need for a controlled carbon-source feed in order to keep carbohydrate concentration in the broth sufficiently low to prevent growth inhibition of the fungus by osmotic effects, thereby simplifying the fermentation process. Furthermore, valorizing remaining sucrose in the pulp through fungal biomass production reduces the economic requirement of optimum sugar extraction, thereby avoiding the relatively high energy costs of extraction of the last remaining sucrose.

In one embodiment, the composition of the invention comprises particles of sugar beet pulp having a d90 value of at most 500 μm. Preferably, the particles of sugar beet pulp have a d90 value of at most 400 μm, preferably at most 300 μm, more preferably at most 200 μm, even more preferably at most 150 μm, even more preferably at most 125 μm, even more preferably at most 100 μm, and most preferably at most 75 μm, and at least 1 μm, preferably at least 2 μm, more preferably at least 5 μm and most preferably at least 10 μm. The particle size distribution, in particular the d90 value, is determined using conventional techniques such as laser diffraction using a Malvern Mastersizer.

In one embodiment of the invention, the inventive composition comprises at least 1 wt % of the sugar beet pulp. Preferably, the inventive composition comprises at least 2 wt % sugar beet pulp, more preferably at least 5 wt % sugar beet pulp, even more preferably at least 10 wt % sugar beet pulp and most preferably at least 15 wt % sugar beet pulp, and preferably at most 60 wt % sugar beet pulp, more preferably at most 50 wt % sugar beet pulp and most preferably at most 40 wt % sugar beet pulp, based on the total weight of the composition.

In one embodiment of the invention, the plant-based fiber may further contain a second plant-based fiber. Examples of suitable second plant-based fibers include brewer spent grain; cereal spent grain; cereal fibers; fruit fiber such as fibers derived from apples and pears; vegetable fibers such as fibers derived from lettuce and legumes such as peas and soybeans; and nuts such as almond. A preferred second plant-based fiber are fibers derived from brewer's spent grain (BSG). Preferably, the coarse fiber composition of BSG, the fine fiber composition of BSG and/or combinations thereof as described in PCT/IB2022/061700. In one embodiment, the plant-based fiber is a coarse fiber composition comprising coarse fibers of cereal grains, preferably BSG, characterized in that the coarse fiber composition comprises at least 65 wt % insoluble high molecular weight dietary fibers (insoluble HMWDF) and less than 15 wt % protein, based on the total dry weight of the coarse fiber composition. Additionally or alternatively, the plant-based fiber is a coarse fiber composition comprising a minimum of 20% by weight of cellulose, a minimum of 40% by weight of hemicellulose and a minimum of 4% by weight of lignin, based on the total dry weight of the coarse fiber composition. In another embodiment, the plant-based fiber is a fine fiber composition comprising fibers of cereal grains, preferably BSG, characterized in that the fine fiber composition is at least 50 wt % and at most 70 wt % insoluble HMWDF, based on the total dry weight of the coarse fiber composition, and wherein the fine fiber composition comprises at most 20 wt % cellulose, based on the total dry weight of the fine fiber composition. Additionally or alternatively, the plant-based fiber is a fine fiber composition comprising at least 40% by weight of hemicellulose and at least 4% by weight of lignin, based on the total dry weight of the fine fiber composition.

Further details and embodiments of the coarse and fine fiber compositions as well as the process to obtain these compositions can be found in PCT/IB2022/061700 and these details and embodiments are included in this specification.

In a further embodiment of the invention, the second plant-based fiber comprises at most 100 ppm gluten. Preferably, the second plant-based fiber comprises at most 50 ppm gluten, more preferably at most 40 ppm gluten, even more preferably at most 30 ppm, and most preferably at most 20 ppm, and preferably at least 10 ppb gluten, more preferably at least 500 ppb, even more preferably at least 1 ppm and most preferably at least 2 ppm. In one embodiment, the second plant-based fiber is gluten-free, i.e. the composition comprises less than 20 ppm. Gluten content was determined using AOAC 2012.01.

In one embodiment, the composition of the invention comprises particles of second plant-based fiber having a d90 value of at most 500 μm. Preferably, the particles of plant-based fiber have a d90 value of at most 400 μm, preferably at most 300 μm, more preferably at most 200 μm, even more preferably at most 150 μm, even more preferably at most 125 μm, even more preferably at most 100 μm, and most preferably at most 75 μm, and at least 1 μm, preferably at least 2 μm, more preferably at least 5 μm and most preferably at least 10 μm. The particle size distribution, in particular the d90 value, is determined using conventional techniques such as laser diffraction using a Malvern Mastersizer.

In one embodiment of the invention, the inventive composition comprises at least 1 wt % of the second plant-based fiber. Preferably, the inventive composition comprises at least 2 wt % second plant-based fiber, more preferably at least 5 wt % second plant-based fiber, even more preferably at least 10 wt % second plant-based fiber and most preferably at least 15 wt % second plant-based fiber, and preferably at most 60 wt % second plant-based fiber, more preferably at most 50 wt % second plant-based fiber and most preferably at most 40 wt % second plant-based fiber, based on the total weight of the composition.

In one embodiment, the fiber-reinforced mycelium filaments comprise an additive. The additive can be any additive known in the art. Such additives include pigments, (inorganic) fillers, flavouring agents, anti-oxidants, sugars, other proteins and colouring agents.

In one embodiment of the invention, the fiber-reinforced mycelium filaments comprise at least 1 wt % of the additive. Preferably, the inventive mycelium filaments comprise at least 2 wt % additive, more preferably at least 5 wt % additive, even more preferably at least 10 wt % additive and most preferably at least 15 wt % additive, and preferably at most 60 wt % additive, more preferably at most 50 wt % additive and most preferably at most 40 wt % additive, based on the total weight of the fiber-reinforced mycelium filaments.

The amounts of mycelium filaments, sugar beet pulp, second plant-based fiber, additives and any other components add up to 100% by weight of the composition of the invention.

The invention further pertains to a food product comprising the fiber-reinforced mycelium filaments comprising fungal mycelium and a plant-based fiber, wherein the plant-based fiber comprises sugar beet pulp. The food product can be any food product known in the art wherein the inventive filaments can be used. Examples of such food products include meat substitutes or alternatives, fish substitutes or alternatives, breakfast cereals, cereal bars, pastry, snacks and salads. Snacks are preferably chosen from the group consisting of plant-based meat snacks, vegan meat sticks, pizza bites and vegan protein bites.

In another embodiment, the food product is a vegetarian or vegan food product, preferably a vegetarian or vegan meat substitute or alternative, fish substitute or alternative, breakfast cereal, cereal bar, pastry, snack or salad. In a preferred embodiment, the food product does not comprise animal-derived ingredients.

In one embodiment of the invention, the food product is a burger, preferably a vegetarian or vegan burger.

The food product can be in any form known in the art. Examples include liquids, such as dispersions, creams, emulsions and solutions, and solids, such as granules, flakes, foams, gels or powders.

In one embodiment of the invention, the food product comprises at least 1 wt % of the fiber-reinforced mycelium filaments. Preferably, the inventive food product comprises at least 2 wt % fiber-reinforced mycelium filaments, more preferably at least 5 wt % fiber-reinforced mycelium filaments, even more preferably at least 10 wt % fiber-reinforced mycelium filaments and most preferably at least 15 wt % fiber-reinforced mycelium filaments, and preferably at most 99 wt % fiber-reinforced mycelium filaments, more preferably at most 90 wt % fiber-reinforced mycelium filaments and most preferably at most 80 wt % fiber-reinforced mycelium filaments, based on the total weight of the food product.

In one embodiment, the food product comprises a food-grade additive. Such a food-grade additive can be any food-grade additive known in the art. Examples of such food-grade additives include flavouring agents, colouring agents, preservatives, proteins, liquids such as water, anti-oxidants and (dietary) fibers.

In one embodiment of the invention, the food product comprises at least 1 wt % of the food-grade additive. Preferably, the inventive food product comprises at least 2 wt % food-grade additive, more preferably at least 5 wt % food-grade additive, even more preferably at least 10 wt % food-grade additive and most preferably at least 15 wt % food-grade additive, and preferably at most 99 wt % food-grade additive, more preferably at most 90 wt % food-grade additive and most preferably at most 80 wt % food-grade additive, based on the total weight of the food product.

The amounts of mycelium filaments, sugar beet pulp, second plant-based fiber, food-grade additives and any other components add up to 100% by weight of the food product.

The invention further pertains to the use of fiber-reinforced mycelium filaments comprising fungal mycelium and a plant-based fiber, wherein the plant-based fiber comprises sugar beet pulp, in food products. The invention further pertains to the use of compositions comprising fiber-reinforced mycelium filaments comprising fungal mycelium and a plant-based fiber, wherein the plant-based fiber comprises sugar beet pulp, in food products

The invention further pertains to a method of producing fiber-reinforced mycelium filaments according to the invention comprising the steps of:
(a) providing an aqueous cultivation medium comprising mycelium filaments and/or fungal hyphae and optionally plant-based fibers, wherein the plant-based fiber comprises sugar beet pulp;
(b) fermenting the mycelium filaments and/or fungal hyphae and optionally adding the plant-based fiber comprising sugar beet pulp during fermentation to obtain a suspension comprising fiber-reinforced mycelium filaments;
(c) deactivating the mycelium filaments and optionally adding the plant-based fiber comprising sugar beet pulp during fermentation;
(d) removing water from the suspension; and
(e) optionally drying the fiber-reinforced mycelium filaments.

The inventive method requires the production of the fiber-reinforced mycelium filaments in the presence of plant-based fiber, in particular sugar beet pulp. In this way, the plant-based fiber forms part of the filamentous structure of the mycelium filaments, which allows for tailoring the physical and/or chemical properties of the mycelium filaments. The presence of the plant-based fiber, in particular sugar beet pulp, may also favourably alter the growing conditions of the mycelium filaments.

In step (a) of the inventive method, an aqueous cultivation medium comprising mycelium filaments and/or fungal hyphae and optionally plant-based fibers, wherein the plant-based fiber comprises sugar beet pulp, is provided. The aqueous cultivation medium comprises water and cultivation agents. Such cultivation agents may be any cultivating agents known in the art and suitable for growing the mycelium filaments and/or fungal hyphae. Such cultivation agents include poly-, di-, monosaccharides and combinations thereof, salts, agar, buffering agents, pH regulating agents. The amounts of the cultivation agents can be chosen as desired by the skilled artisan. It is noted that the sugar beet pulp may be consumed during fermentation and that therefore the amount of cultivation agents may be reduced or even discarded. In one embodiment, part of the plant-based fiber, preferably sugar beet pulp, is present before the fermentation step.

In one embodiment, the aqueous cultivation medium comprises water in an amount of at least 10% by weight (wt %), based on the total weight of the aqueous cultivation medium. Preferably, water is present in an amount of at least 15 wt %, more preferably at least 20 wt %, even more preferably at least 30 wt % and most preferably at least 40 wt %, and preferably at most 99 wt %, more preferably at most 98 wt %, even more preferably at most 95 wt % and most preferably at most 90 wt %, based on the total weight of the suspension.

The mycelium filaments and/or fungal hyphae can be any mycelium filaments or fungal hyphae known in the art which can be suitably used in the method if the invention. Suitable fungal mycelium and fungal hyphae and their embodiments have been described above. The mycelium filaments and/or fungal hyphae may be present in an amount of at least 0.001% by weight (wt %), based on the total weight of the aqueous cultivation medium. Preferably, the mycelium filaments and/or fungal hyphae is present in an amount of at least 0.01 wt %, more preferably at least 0.1 wt %, even more preferably at least 0.5 wt % and most preferably at least 1 wt %, and preferably at most 15 wt %, more preferably at most 10 wt %, even more preferably at most 8 wt % and most preferably at most 5 wt %, based on the total weight of the suspension.

The plant-based fibers comprises sugar beet pulp and may comprise a second plant-based fiber which can be any plant-based fibers known in the art which can be suitably used in the method if the invention. Suitable second plant-based fibers and their embodiments have been described above. The plant-based fiber, preferably the sugar beet pulp, may be present in an amount of at least 0.1% by weight (wt %), based on the total weight of the aqueous cultivation medium. Preferably, the plant-based fiber is present in an amount of at least 0.5 wt %, more preferably at least 1 wt %, even more preferably at least 1.5 wt % and most preferably at least 2 wt %, and preferably at most 30 wt %, more preferably at most 25 wt %, even more preferably at most 20 wt % and most preferably at most 10 wt %, based on the total weight of the suspension.

In another preferred embodiment, the pH of the cultivation medium in step (a) is at most 8, more preferably at most 7, and most preferably at most 6, and preferably at least 4, more preferably at least 4.5, and most preferably at least 5. It is envisaged to adjust the pH during step (a) when necessary.

The temperature of the cultivation medium during step (a) is maintained at a temperature above 0° C. Preferably, the temperature of the cultivation medium is at least 5° C., more preferably at least 10° C., more preferably at least 15° C. and most preferably at least 20° C., and preferably at most 35° C., more preferably at most 30° C., and most preferably at most 25° C. Preferably, the temperature of the cultivation medium in step (a) is chosen to avoid proliferation of the fungal hyphae and/or mycelium filaments. Alternatively, the temperature can be chosen higher, and even as high as the temperature at which the fermentation step (b), when the cultivation medium and/or its ingredients have been sterilized before and/or during step (a).

In step (b) of the inventive method, the mycelium filaments and/or fungal hyphae are fermented to obtain a suspension comprising fiber-reinforced mycelium filaments. In one embodiment, the fermentation is a submerged fermentation. In one embodiment, the plant-based fiber, preferably the sugar beet pulp, may be added continuously or intermittently during fermentation. In another embodiment, part of the plant-based fibers, in particular the sugar beet pulp, is added at the end of the fermentation step (b), preferably just before the inactivation step (c). In this way, the fibers, preferably the sugar beet pulp, will remain intact (i.e. are hardly consumed during fermentation) so that the physical and chemical properties of the plant-based fiber is not altered significantly.

In a preferred embodiment, the pH of the suspension in step (b) is at most 8, more preferably at most 7, and most preferably at most 6, and preferably at least 4, more preferably at least 4.5, and most preferably at least 5. It is envisaged to adjust the pH during step (b) when necessary. Preferably, the pH of the suspension in step (b) is the same as the temperature of the suspension in step (a).

The temperature of the suspension during step (b) is maintained at a temperature above room temperature. Preferably, the temperature of the suspension is at least 10° C., more preferably at least 15° C., more preferably at least 20° C. and most preferably at least 25° C., and preferably at most 160° C., more preferably at most 100° C., even more preferably at most 70° C., and most preferably at most 60° C.

In step (c) of the inventive method, the fiber-reinforced mycelium filaments are deactivated. This deactivation prevents the formation of mycotoxins and/or undesirable RNA fragments. Generally, deactivation of the mycelium filaments can be achieved by a heat treatment, treatment with steam or an acidic treatment. Conditions used for pasteurization or sterilization of food products may be suitable for step (c). The plant-based fiber, preferably comprising the sugar beet pulp, is added in this step (c), which constitutes a physical mixture of the filaments and the plant-based fiber resulting in the fiber-reinforced mycelium filaments of the invention

In step (d), water can be removed from the suspension comprising the fiber-reinforced mycelium filaments using any method known in the art. Such methods include filtration, microfiltration, ultrafiltration, pressing, decantation and evaporation.

In optional step (e), the fiber-reinforced mycelium filaments obtained in step (d) are dried. Drying can be performed using conventional techniques such as fluidized bed drying or spray drying. When the fiber-reinforced mycelium filaments are dried further, dried powder or particles can be obtained.

In one embodiment, water may be present in an amount of at most 10% by weight (wt %), based on the total weight of the fiber-reinforced mycelium filaments. Preferably, the fiber-reinforced mycelium filaments are present in an amount of at most 9 wt %, more preferably at most 7 wt %, even more preferably at most 6 wt % and most preferably at most 5 wt %, and preferably at least 0.1 wt %, more preferably at least 0.5 wt %, even more preferably at least 1 wt % and most preferably at least 2 wt %, based on the total weight of the fiber-reinforced mycelium filaments.

The invention is exemplified in the following Examples.

### EXAMPLES

### Examples 1 and 2: Mycelium Filaments with Sugar Beet Fiber

### Inoculum Preparation

A fungal spore suspension of Rhizopus oryzae was prepared by flooding PDA plate cultures with 10 mL sterile water and scraping spores off the surface with a disposable, sterile spreader. Obtained suspensions were either used to inoculate bioreactor cultivations, or to inoculate pre-culture: 200 mL YPD medium (10 g/L yeast extract, 20 g/L bacto peptone, 20 g/L glucose) in 500 mL baffled Erlenmeyer flasks. Pre-cultures were incubated for 18-24 h at 30° C. shaking at (150 RPM).

Bioreactor Cultivations with Sugar Beet Pulp and Whole Sugar Beet

Frozen sugar beet pulp, sampled from Cosun Beet Company's Dinteloord plant, was thawed and size-reduced to approximately 1-4 mm using a Kenwood kitchen blender to enable homogenous suspension in a stirred lab-fermenter. Fresh sugar beet was peeled and similarly size-reduced.

200 g of either whole sugar beet or sugar beet pulp was added to 980 mL demineralized water in Infors Labfors 2 L fermentation vessels equipped with 2 rushton- and 1 marine impellers and sterilized for 20 min at 121° C. A third vessel was prepared without beet material to serve as reference. After cooling, 20 mL sterile 50× Vogel's minimal medium (modified from Microbial Genetics Bulletin 13:42-43, 1956: 1 g/L (NH₄)₂SO₄ as (batch-phase) N-source, (start) pH 6.0, no chloroform) stock solution was added. To vessels containing sugar beet pulp or no beet material, 5 g sucrose per L of liquid medium was added.

Bioreactor cultivations were inoculated with 10 mL of Rhizopus oryzae (CBS128.08 obtained from Westerdijk Institute, Utrecht, the Netherlands) spore suspension harvested from plate cultivations. Cultivations were run for 90 hours at 30° C., aerated at a rate of 1 VVM with dissolved oxygen controlled to a minimum of 30% saturation by a stirrer-cascade (400-1500 RPM), pH was maintained at 6.0 by 2-sided pH control (12.5% (m/m) NH₄OH, 10% (m/m) H₂SO₄). Upon CPR (carbon dioxide production rate) decrease, vessels were fed with 35% (m/m) glucose with feed rate controlled by dissolved oxygen level (agitation fixed at 750 RPM).

Scanning electron-microscopy (SEM) analyses of mycelium obtained from these cultivations at 90 h clearly show that whereas under reference conditions (no beet material present) the fungus forms a mycelial network. When provided with beet material, the fungal hyphae associate with and attach to the fiber structures. In FIG. 1, it is indicated (with arrows) that in both examples (sugar beet fiber (SBP on left; Example 1) and whole sugar beet (whole beet on right; Example 2)) fungal hyphae not only attach to, but also penetrate the beet structures both through beet cell walls and intercellular spaces. This results in a fungus-beet filament structure in which the hyphae are effectively supported (reinforced) by the beet fiber.

### Examples 3 and 4: Different Organisms on Sugar Beet Pulp

### Inoculum Preparation

Fungal biomass of Pleurotus pulmonarius (CBS 507.85 obtained from Westerdijk Institute, Utrecht, the Netherlands) was prepared by taking a MEA (malt extract agar) plate culture and scraping biomass off the surface with a disposable, sterile loop. Obtained biomass of Pleurotus pulmonarius and 1 mL stock culture (−80° C., 25% m/m glycerol) of Aspergillus oryzae (CBS 466.91 obtained from Westerdijk Institute, Utrecht, the Netherlands) were used to inoculate the pre-cultures: 200 mL Vogel's minimal medium supplemented with 20 g/L glucose in 500 mL baffled Erlenmeyer flasks. Pre-cultures were incubated for 24 h at 24° C., shaken at 150 RPM and subsequently used to inoculate the bioreactor cultivations.

Bioreactor Cultivations with Sugar Beet Pulp

Frozen sugar beet pulp, sampled from Cosun Beet Company's Dinteloord plant, was thawed, washed and size-reduced to approximately 1-4 mm using a Kenwood kitchen blender to enable homogenous suspension in a stirred lab-fermenter.

147 g of sugar beet pulp was added to 773 mL demineralized water in Eppendorf SciVario twin 2 L fermentation vessel equipped with 3 rushton impellers and sterilized for 20 min. at 121° C. After cooling, 20 mL sterile 50× Vogel's minimal medium stock solution was added. Additionally, 10 mL of a 50% (m/v) glucose solution was supplemented to the vessels.

Bioreactor cultivations were inoculated with 50 ml of Pleurotus pulmonarius or Aspergillus oryzae suspended biomass harvested from Vogel's minimal medium pre-culture cultivations. Cultivations ran for 257 and 112 hours respectively at 24° C., with dissolved oxygen controlled to a minimum of 30% saturation by a stirrer (800-1500 RPM) and aeration (0.5-1.5 VVM) cascade, pH was maintained at 6.0 by 2-sided pH control (10% (m/m) KOH, 10% (m/m) H₂SO₄). Upon CPR (carbon dioxide production rate) decrease, vessels were fed with 50% (m/m) glucose with feed rate controlled by dissolved oxygen level.

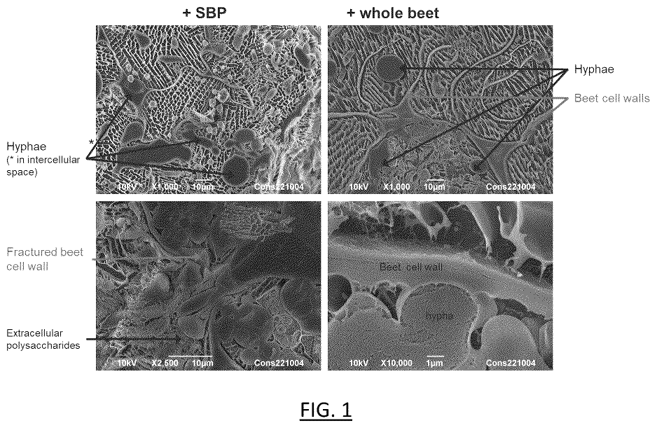

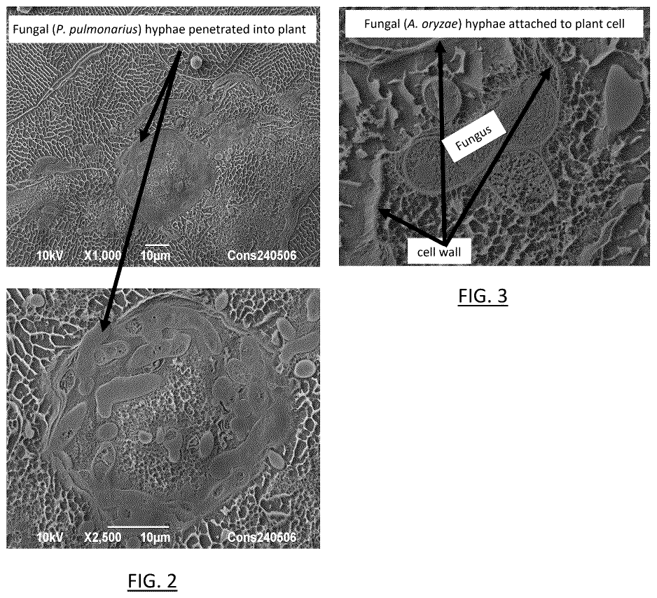

Scanning electron-microscopy (SEM) analyses of mycelium obtained from these cultivations at the end of fermentation clearly show that the fungal hyphae of Pleurotus pulmonarius (Example 3) and Aspergillus oryzae (Example 4) associates with—and attaches to the fiber structures of the sugar beet pulp. In FIG. 2, it is indicated (with arrows) that P. pulmonarius hyphae not only attaches to, but also penetrates the sugar beet structures both through beet cell walls and intercellular spaces. FIG. 3 indicates (with arrows) the plant cell wall and in the middle the fungal hyphae attached to the plant cell. Both results in a fungus-beet filament structure in which the hyphae are effectively supported (reinforced) by the beet fiber.

### Examples 5 and 6: Physical Properties of Mycelium Filaments with Sugar Beet Pulp

### Inoculum Preparation

A fungal spore suspension of Rhizopus oryzae (CBS 285.55 obtained from Westerdijk Institute, Utrecht, the Netherlands) was prepared by flooding a MEA (malt extract agar) plate culture with 10 mL sterile water and scraping spores off the surface with a disposable, sterile spreader. Obtained suspensions were used to inoculate the pre-culture: 200 mL Vogel's minimal medium supplemented with 20 g/L glucose in 500 mL baffled Erlenmeyer flasks. Pre-cultures were incubated for 48 h at 27° C., shaken at 150 RPM, and subsequently used to inoculate the bioreactor cultivations.

Bioreactor Cultivations with Sugar Beet Pulp

Frozen sugar beet pulp, sampled from Cosun Beet Company's Dinteloord plant, was thawed, washed and size-reduced to approximately 1-4 mm using a Kenwood kitchen blender to enable homogenous suspension in a stirred lab-fermenter.

180 g of sugar beet pulp was added to 770 mL demineralized water in Eppendorf SciVario twin 2 L fermentation vessel equipped with 3 rushton impellers and sterilized for 20 min at 121° C. A second vessel was prepared with 950 mL demineralized water without beet material to serve as reference. After cooling, 40 mL sterile 50× Vogel's minimal stock solution was added. Additionally, 10 mL of a 50% (m/v) glucose solution was supplemented to the vessels.

Bioreactor cultivations were inoculated with 50 mL of Rhizopus oryzae suspended biomass harvested from Vogel's minimal medium pre-culture cultivations. Cultivations ran for 144 hours at 27° C., with dissolved oxygen controlled to a minimum of 30% saturation by a stirrer (200-1500 RPM) and aeration (0.5-1.5 VVM) cascade, pH was maintained at 6.0 by 2-sided pH control (10% (m/m) KOH, 10% (m/m) H₂SO₄). Upon CPR (carbon dioxide production rate) decrease, vessels were fed with 50% (m/m) glucose with feed rate controlled by dissolved oxygen level.

The reinforced mycelium obtained in the presence of sugar beet pulp is referred to as Example 5. A further sample was prepared where an aqueous mycelium suspension (9.28 wt % dry matter) was physically mixed with sugar beet pulp (13.48 wt % dry matter) in a 1:1 weight ratio to obtain a physical mixture with 7.67 wt % dry matter (Example 6). Next to these Examples the sugar beet pulp (Comparative Example A) and the mycelium per se as obtained above (Comparative Example B) were used in the characterization methods described below.

### Penetration Test

The examples were diluted to a dry matter content of 7.67 wt % and characterized using a Brookfield CT3 texture Analyzer and a ball-shaped probe (diameter 6 mm). The probe is pressed in the samples at a constant speed of 0.5 mm/s until a depth of 15 mm is reached. The peak force and peak area (area under the force-deformation curve). The results are shown in the Table below.

TABLE 1
Peak force and peak area

| Example | Peak force (g) | Peak area (mJ) |
|---|---|---|
| Comparative Example A | 34.5 | 3.11 |
| Comparative Example B | n.d. | n.d. |
| Example 5 | 3.0 | 0.1 |
| Example 6 | 27 | 2.4 |

n.d. not determined

The re-inforced mycelium of Example 5 has a lower peak force and peak area compared to beet pulp as such (Comparative Example A) and the physical mixture of sugar beet pulp and R. oryzae filaments (Example 6). The mixture of Example 6 has a lower peak force and peak area then Comparative Example A.

The examples were centrifuged at 2460 rpm for 10 minutes at room temperature. The amount of supernatant and pellet were determined, and shown in the Table below.

TABLE 2
Amount of supernatant and pellet after centrifugation

| | Supernatant | Pellet |
|---|---|---|
| Example | (wt %) | (wt %) |
| Comparative Example A | 28 | 72 |
| Comparative Example B | 65 | 35 |
| Example 5 | 76 | 24 |
| Example 6 | 35 | 65 |

The reinforced mycelium of Example 5 clearly has a different pelletization behaviour than the physical mixture of Example 6 and the beet pulp of Comparative Example A. A higher supernatant amount and a lower pellet amount were observed for Example 5 compared to the filaments per se (Comparative Example B). This means that the fiber-reinforced mycelium of Example 5 holds water to a lower extent than the mycelium per se and forms a denser pellet. The physical mixture of Example 6 appears to hold water better than the mycelium per se and forms a looser pellet. A skilled person will appreciate that by fermentation with sugar beet pulp and adding sugar beet pulp at the end of or after the fermentation step the properties of the fiber-reinforced mycelium can be altered as desired.

### Example 7: Mycelium Filaments with Sugar Beet Pulp

### Inoculum Preparation

A fungal spore suspension of Rhizopus oryzae (CBS 285.55 obtained from Westerdijk Institute, Utrecht, the Netherlands) was prepared by flooding a MEA (malt extract agar) plate culture with 10 mL sterile water and scraping spores off the surface with a disposable, sterile spreader. Obtained suspensions were used to inoculate the pre-culture: 200 mL Vogel's minimal medium supplemented with 20 g/L glucose in 500 mL baffled Erlenmeyer flasks. Pre-cultures were incubated for 48 h at 27° C., shaken at 150 RPM, and subsequently used to inoculate the shake flask cultivations with sugar beet pulp (as such).

Bioreactor Cultivations with Sugar Beet Pulp

Frozen sugar beet pulp, sampled from Cosun Beet Company's Dinteloord plant, was thawed and washed with demineralized water. 18 g sugar beet pulp (as such) was added to 74 mL demineralized water in 500 mL baffled Erlenmeyer flasks and sterilized for 20 min at 121° C. After cooling, 4 mL sterile 50× Vogel's minimal stock solution was added. Additionally, 4 mL of a 50% (m/v) glucose solution was supplemented to the shake flask.

The shake flask cultivation was inoculated with 5 mL of Rhizopus oryzae suspended biomass harvested from Vogel's minimal medium pre-culture cultivations. Cultivations ran for 42 hours at 27° C., shaken at 150 RPM.

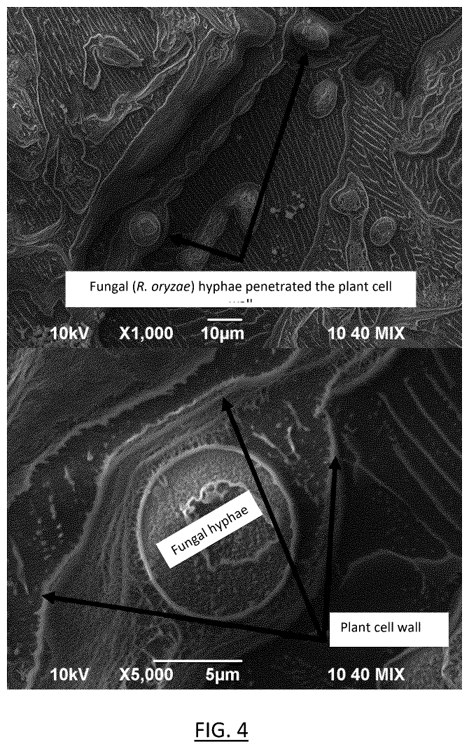

Scanning electron-microscopy (SEM) analyses of mycelium obtained from this cultivation at 44 h (Example 7) clearly show that the fungal hyphae associate with and attach to the fiber structures. In FIG. 4, it is indicated (with arrows) that the fungal hyphae not only attach to, but also penetrates the sugar beet structures both through plant (beet) cell walls. This results in a fungus-beet filament structure in which the hyphae are effectively supported (reinforced) by the beet fiber.

1. Composition comprising fiber-reinforced mycelium filaments comprising fungal mycelium and a plant-based fiber, wherein the plant-based fiber comprises sugar beet pulp.

2. Composition according to claim 1 comprising at most 50 wt % of plant-based fiber, based on the total dry weight of the fiber-reinforced mycelium filament.

3. Composition according to claim 1, wherein the fungal mycelium is a fungal mycelium selected from the group consisting of of Rhizopus oryzae, Aspergillius oryzae and Pleurotus pulmonarius.

4. Composition according to claim 1, wherein the plant-based fiber has a d90 of at most 500 mm.

5. A food product comprising the composition according to claim 1.

6. The food product according to claim 5, wherein the product is a vegan burger.

7. (canceled)

8. (canceled)

9. Method of producing fiber-reinforced mycelium filaments comprising fungal mycelium and a plant-based fiber, wherein the plant-based fiber comprises sugar beet pulp comprising the steps of:
(a) providing an aqueous cultivation medium comprising mycelium filaments and/or fungal hyphae and optionally plant-based fiber, wherein the plant-based fiber comprises sugar beet pulp;
(b) fermenting the mycelium filaments and/or fungal hyphae and optionally adding the plant-based fiber comprising sugar beet pulp during fermentation to obtain a suspension comprising fiber-reinforced mycelium filaments;
(c) deactivating the fiber-reinforced mycelium filaments in the suspension, and optionally adding the plant-based fiber comprising sugar beet pulp;
(d) removing water from the suspension; and
(e) optionally drying the fiber-reinforced mycelium filaments.
